(19) United States
(12) Patent Application Publication
(10) Pub. No.: US 2026/0091901 A1
(43) Pub. Date: Apr. 2, 2026

(54) A METHOD FOR STERILISING A COMPONENT OF A MACHINE OPERATING IN A STERILE CLOSED ENVIRONMENT AND USED FOR CONTACT WITH PRODUCTS WHICH ARE TO BE PROCESSED INTERNALLY OF THE CLOSED ENVIRONMENT

(71) Applicant: MARCHESINI GROUP S.P.A., PIANORO (BO) (IT)

(72) Inventor: Simone NANNETTI, GRIZZANA MORANDI (BO) (IT)

(73) Assignee: MARCHESINI GROUP S.P.A., PIANORO (BO) (IT)

(21) Appl. No.: 19/110,418

(22) PCT Filed: Sep. 7, 2023
(86) PCT No.: PCT/IB2023/058858

§ 371 Date: Mar. 10, 2025

(30) Foreign Application Priority Data
Sep. 12, 2022 (IT) 102022000018513

(51) Int. Cl. B65B 55/04; A61L 2/07; B65B 55/02

(52) U.S. Cl. CPC B65B 55/04; A61L 2/07; B65B 55/027; A61L 2202/121; A61L 2202/122; A61L 2202/17

(57) ABSTRACT

The method for sterilising a component of a machine operating in a sterile closed environment, and used for contact with products which are to be processed internally of the closed environment, comprises: sealedly wrapping the first part of the component, destined for contact with the products, internally of a first envelope having a hole at which the second part of the component remains exposed and accessible, which second part is destined to be coupled to a the base or frame present internally of the closed environment, and to wrap the component, partially wrapped in the first envelope, internally of a second envelope and to close the second envelope, and then carry out a sterilisation cycle.

### FIELD OF THE INVENTION

The present invention relates to the special technical sector regarding the processing and/or packaging of products, such as, for example, but not exclusively, pharmaceutical products in liquid or paste form or in the form of tablets or capsules, and the like, in sterile and aseptic conditions, and which are to carried out internally of machines or apparatuses that are designed to comprise suitable closed operating environments (or chambers) free of contamination.

These machines must be designed in such a way as to comprise closed working environments internally of which specifications must be observed in order to maintain them in completely aseptic and sterile conditions, for example with the use of laminar flows and appropriate air filtering systems, so as to prevent any possible source of contamination coming from the external environment.

For example, the closed operating environment internally of which the strictest aseptic and sterile conditions must be maintained is normally classified, on the basis of existing standards, with the letter “A”, while the contiguous and external environments are classified with successive letters of the alphabet, such as, for example, “B”, “C” and “D”, progressively as the distance from the sterile ambient increases and the aseptic and sterile conditions decay, as contamination sources are present.

In these particular types of machines (such as, for example, isolators, the machines known by the terms oRABS or cRBAS), internally of the class “A” closed operating environment, i.e. sterile and aseptic, a series of components is present which components are destined to go into contact with the products that are to be processed.

For example, for the packaging of pharmaceutical products, internally of relative containers, such as for example bottles, vials, syringes, tubes, and the like, supply nozzles or other supply devices can be used for transferring the products to inside the containers, such as hoppers, internally of which collars and/or caps are stored for closing the containers once filled, as well as vibrating cups into which the caps are unloaded, or other closing elements, for the successive supply thereof to the devices for collecting and application of the caps, or of the closing elements, to the containers.

### DESCRIPTION OF THE PRIOR ART

Usually, these components comprise at least a first part, which is destined for direct contact with the products, and a second part which is destined to be coupled to a base, or to a frame, present internally of the closed operating environment, on which the operating means provided and responsible for the driving and operating thereof are mounted.

For example, a vibrating cup comprises a first part, which is conformed substantially as a bowl and comprising a track or conveying channel of the caps, which is destined to receive the caps internally thereof, and a second part, below the first part, which is destined to be coupled to a base responsible for the driving in rotation and vibration of the cup.

In this particular type of machines, when it is necessary to change the product to be processed or after a period of machine inactivity, which has led to the loss of the sterile and aseptic conditions internally of the closed operating environment, there exists the problem of how to carry out the sterilisation of the components destined for direct contact with the products to be processed, and then the successive re-insertion and remounting thereof internally of the closed operating environment, preventing the parts thereof destined for direct contact with the products from being contaminated or from coming into contact with sources of contamination.

Regarding this, a prior art method includes demounting the components which are present internally of the closed operating environment in class “A” and carrying them, through special openings or windows, to outside the closed environment, to an external environment (usually in class “D”) where special washing machines are present to carry out the washing (see for example FIG. 1A).

Then, the components, once washed, are closed internally of a special envelope which is then closed and sealed.

The component closed internally of an envelope of this type is brought into a second more sterile environment, in class “C”; with respect to the environment in which the washing machines operate, and inserted in special autoclaves for the process of sterilisation (see for example FIG. 1B).

The envelope in which the component is sealed is made of a material suitable for allowing the passage of the sterilisation fluid and for maintaining sterile conditions internally of the envelope to avoid external contaminations.

Then, once the process of sterilisation has been completed, the component will be inside the envelope in an environment that is completely sterile and isolated, by the envelope, from the outside environment.

Following this, the closed envelope is transferred, with the sterilised component inside, either to a class “B” environment contiguous to the class “A” closed environment (see FIG. 1C), or to an outside environment in class “C”, and from there inserted internally of the class “A” closed environment, or even directly internally of the closed operating environment of the machine.

The envelope is then cut and removed from the component and the component remounted on the relative base or frame.

These operations can be carried out from the outside, through special windows or passages present in a wall of the machine, or with the machine open and with an operator entering the closed operating environment.

These procedures, inevitably, can expose the parts of the component, destined for direct contact with the products to be processed, to possible contaminations with a consequent decaying of the classification of the closed operating environment.

At present, once the component has been remounted, internally of the closed operating environment a sterilisation cycle is characterised by means of a flow of hydrogen peroxide in the form of steam (known as the VHP cycle) in order to sterilise all the exposed parts of the component.

At present, carrying out a VHP cycle is considered effective as a sterilisation method of the components remounted internally of the closed operating environment and thus effective for restoring internally thereof an “A” type classification, as regards aseptic and sterile conditions.

Now, in the near future, with new standards being introduced in this particular technical sector, the execution of a VHP cycle will no longer be considered sufficient to restore the conditions of sterility with the aim of having an “A” classification internally of the closed operating environment.

The problem therefore arises of how to carry out the sterilisation operations of the components and the successive re-insertion and remounting thereof internally of a closed operating environment of a machine operating in sterile and aseptic conditions so as to avoid the parts of the component destined to come into direct contact with the products being subject to contaminations, such as to preclude the use thereof in a class “A” environment, as the execution of a VHP cycle will no longer be considered sufficient to restore the suitable conditions of sterility on the parts that may be exposed to contamination.

### SUMMARY OF THE INVENTION

The aim of the present invention is therefore to provide a new method for sterilising a component of a machine operating in a sterile closed environment and used for contact with products that are to be processed internally of the closed environment, able to obviate the drawback.

In particular, an aim of the invention is to provide a method that enables carrying out the sterilisation of the components in contact with the products that are to be processed internally of a sterile closed environment and to enable the remounting thereof internally of the sterile and closed operating environment, protecting the parts thereof destined for contact with the products from external contaminations.

The above-mentioned advantages are obtained according to the contents of the claims.

### BRIEF DESCRIPTION OF THE DRAWINGS

The characteristics of the invention will clearly emerge from the following description of a preferred, but not exclusive, embodiment of the mode of execution of the sterilisation method carried out with reference to the appended tables of drawings, in which:

FIGS. 1A, 1B and 1C, already mentioned in the foregoing, illustrate the arrangement of various ambients having different contamination classification which are located externally and about a closed operating environment having an “A” classification and internally of which processing and packaging operations are to be carried out on products in sterile and aseptic conditions;

FIGS. from 2A to 2I schematically illustrate a preferred operating sequence of the sterilisation method proposed by the invention;

FIG. 3A, and 3B illustrate, in respective views in vertical section, possible modalities of execution of a step of the method of the invention;

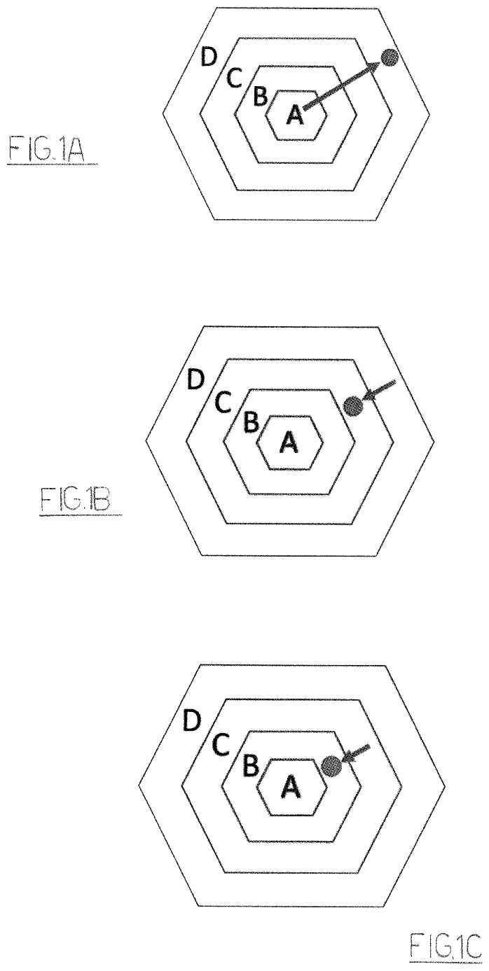

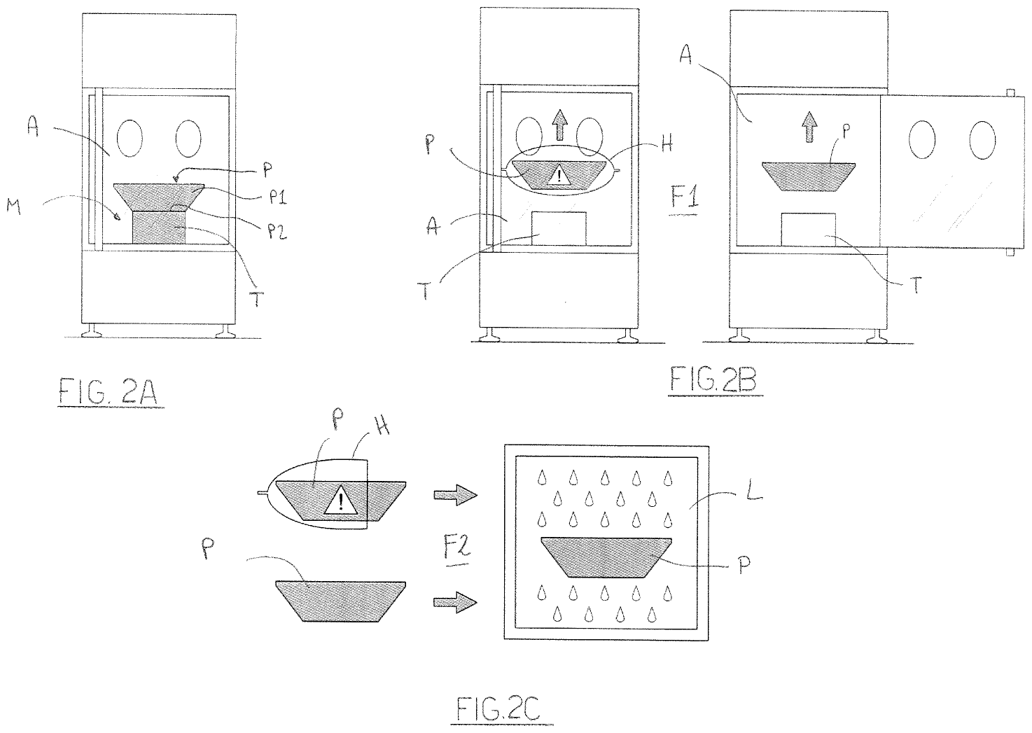

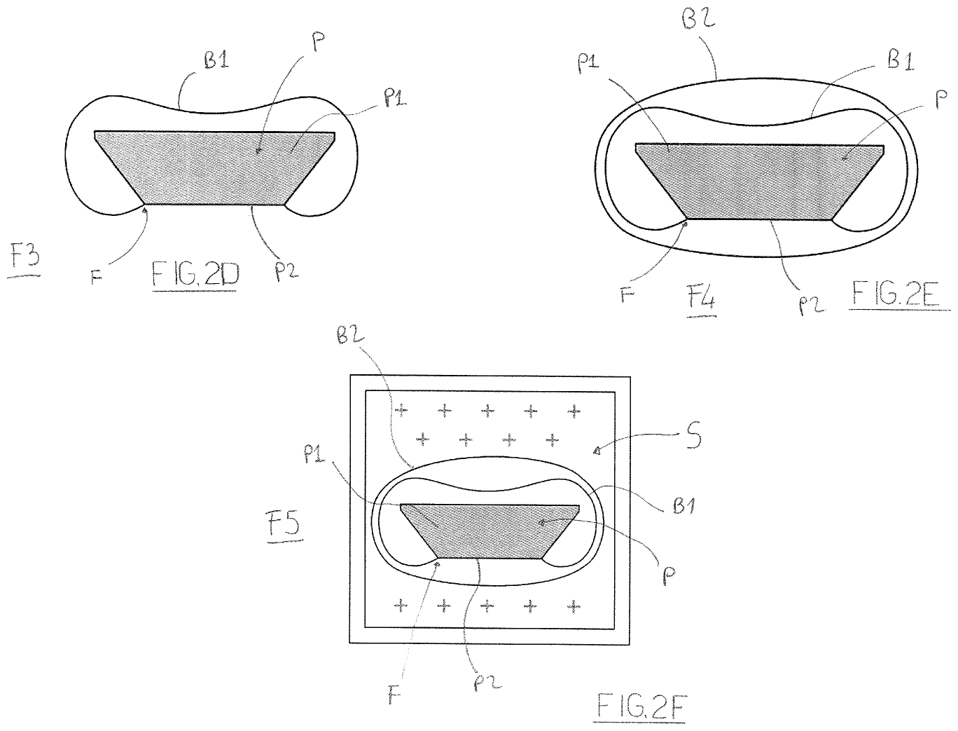

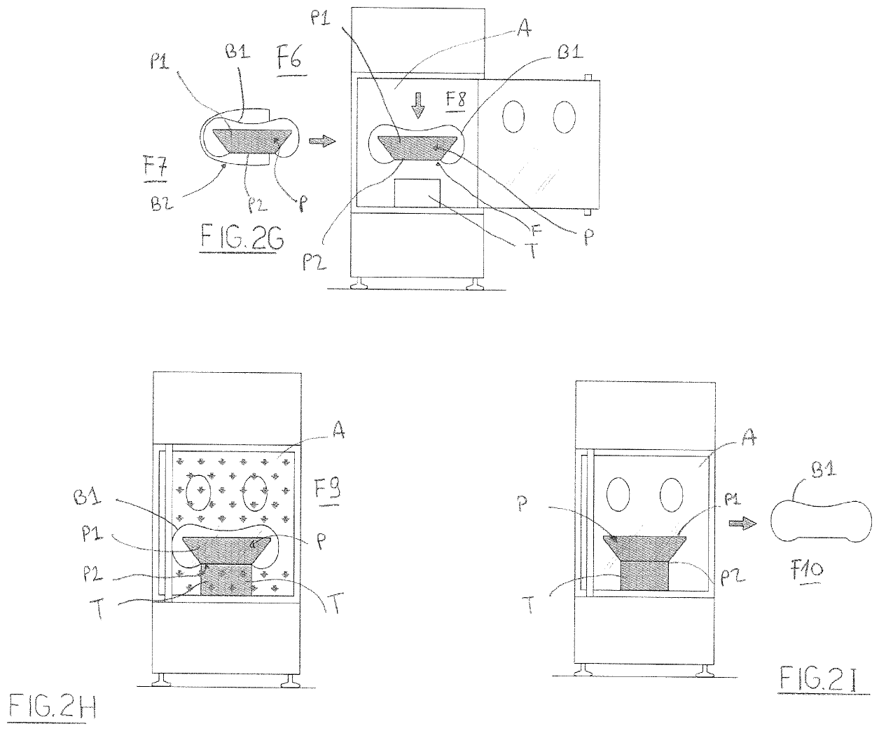

FIGS. from 4A to 4D are schematic views of application of some steps of the method of the invention for sterilisation of a vibrating cup;

FIGS. from 5A to 5C are schematic views of application of some steps of the method of the invention for sterilisation of a hopper;

FIGS. from 6A to 6C are schematic views of application of some steps of the method of the invention for sterilisation of a supply head with dispensing nozzles of liquid.

### DESCRIPTION OF PREFERRED EMBODIMENTS

FIG. 2A schematically illustrates, a machine (M) situated internally of a closed environment (A) which is maintained in sterile and aseptic conditions, for example by means of laminar flows and the use of appropriate filters, in which packaging or processing operations are to be carried out on products with the use of components (P), for example filling of containers with pharmaceutical products, and then the closing of the containers using closing elements, such as, for example, caps.

FIG. 2A also schematically illustrates, a component (P) which comprises a first part (P1), destined for direct contact with the products to be processed internally of the sterile closed environment (A), and at least a second part (P2), which is instead conformed to be mounted and coupled on a base (T) or frame (T) of the machine (M) present internally of the closed environment (A) and comprising respective driving means for driving and operating the component (P).

For example, the component (P) can consist of a cup predisposed to receive, internally thereof, the caps which are to be used for closing containers once filled with a respective product, and which is set in vibration to enable the supplying in line of the caps to a collecting point of the cup.

The cup will comprise a first part, destined to receive the caps, and a second part which is instead destined to be mounted on a base present internally of the closed environment, and comprising suitable means for driving the cup in vibration.

The method of the invention has the application of carrying out the sterilisation of all those components which are used for processing products internally of a sterile closed environment and which are realised in such a way as to comprise a first part, destined for direct contact with the products to be processed, and a second part which is instead destined to be mounted and coupled on a base or frame, present internally of the closed environment, comprising means for driving the components.

In this regard, by way of example,-figures from 4A to 4D, from 5A to 5C and from 6A to 6C, illustrate examples of the method on three types of components which can be used internally of a sterile closed environment for filling containers, such as a vibrating cup (FIGS. 4A-4D), a hopper (FIGS. 5A-5C), and a supply head with dispensing nozzles of liquids and/or pasty substances (FIGS. 6A-6C).

With reference to figures from 2A to 2I, the following contains the main and special steps of the method of the invention.

The method comprises:
a step of demounting (F1) the component (P) from the base (T) or from the frame (T) of the machine (M) present internally of the closed environment (A) (FIG. 2B);
a first step of transferring (F2) the component (P) to outside the sterile closed environment (A) and inserting the component (P) internally of a washing machine (L) in order to carry out a washing cycle of the component (P) (FIG. 2C).

Once the washing cycle has finished, the method comprises:
a first step of wrapping (F3) of the component (P) with a first envelope (B1) comprising completely wrapping the first part (P1) of the component (P) destined for direct contact with the products leaving the second part (P2) of the component (P), destined for coupling and mounting on a base (T) or frame (T) present internally of the closed environment (A), exposed and accessible via a hole (F) present in the first envelope (B1), wherein the first step of wrapping (F3) comprises coupling the edges of the hole (F) sealingly to a portion of the component (P) between the first part (P1) and the second part (P2) of the component (P) to isolate the first part (P1) of the component (P) from the outside environment (FIG. 2D);
a second step of wrapping (F4) comprising completely wrapping the first envelope (B1), and the second part (P2) of the component (P) which remains exposed from the first envelope (B1), with a second envelope (B2) in such a way that the first envelope (B1), and the second part (P2) of the component which is exposed and accessible from the first envelope (B1) via the hole (F), are completely closed internally of the second envelope (B2), with the second envelope (B2) being closed (FIG. 2E);
a second step of transferring (F5) of the component (P), partially wrapped in the first envelope (B1) and completely contained in the second closed envelope (B2), internally of the sterilisation machine(S) in order to carry out a sterilisation cycle by means of a sterilisation flow, wherein the first envelope (B1) and the second envelope (B2) are made of a material capable of allowing passage of the sterilisation flow in such a way that the sterilisation flow can sterilise both the second part (P2) of the component (P), exposed and accessible via the hole (F) of the first envelope (B1), and arranged internally of the second envelope (B2), and also the first part (P1) of the component (P) closed internally of the first envelope (B1) (FIG. 2F).

Then, once the sterilisation cycle has finished, the method comprises (see FIG. 2G):
a third step of transferring (F6) the component (P) with the first part (P1) wrapped in the first envelope (B1) and completely closed in the second envelope (B2) in a position facing the sterile closed environment (A);
a step of cutting and removal (F7) of the second envelope (B2);
a fourth step of transferring (F8) the component (P), with the first envelope (B1) still being sealedly wrapped about the first part (P1) of the component (P) and with the second part (P2) of the component (P) exposed and accessible via the hole (F) of the first envelope (B1), internally of the sterile closed environment (A), and mounting the second part (P2) of the component (P) on the base (T) or frame (T) present internally of the sterile closed environment, maintaining the first part (P1) of the component sealedly closed in the first envelope (B1).

Lastly, the method comprises:
a step of cutting and removal (F10) of the first envelope (B1) from the first part (P1) of the component (P) (FIG. 21).

Owing to the use of the two envelopes, in particular of the first envelope (B1) which is sealedly wrapped about the first part (P1) of the component (P), destined for direct contact with the products to be processed internally of the sterile closed environment (A), and which comprises a hole (F) by means of which the second part (P2) of the component (P) is accessible, with the method of the invention it is possible, once the sterilisation step has been carried out, to proceed to the removal of the second envelope and to the mounting of the component internally of the closed environment, while maintaining the first part of the component sealedly closed in the first envelope and therefore insulated from the outside environment, thus protecting it from any possibility of contamination.

Therefore, with the method of the invention, the first part of the component, i.e. the part destined for direct contact with the products to be processed internally of the sterile closed environment, remains always protected and sealedly closed internally of the first envelope, for all the time necessary for carrying out the remounting of the component on the relative base or frame.

This also considerably facilitates the operations that are to be carried out for remounting the component, which can be carried out even with the machine open, as the first part of the component remains always closed and sealedly protected internally of the first envelope, or enables the mounting thereof even with the machine closed using the special openings included for the insertion of an operator's hands or arms covered with appropriate protective gloves.

Further and other preferred aspects of the method of the invention are described in the following.

In a first preferred aspect, the method comprises using a first seal ring (1) and a second seal ring (2) and applying the first seal ring (1) and the second seal ring (2) on opposite sides of the hole (F) to clamp there-between portions of the first envelope (B1) which surround the hole (F).

In particular, the method comprises using a first seal ring (1) which is conformed in such a way as to be sealedly couplable (for example by interpositioning of a first elastic element) to a portion (10) of the component between the first part (P1) of the component (P) and the second part (P2) of the component, and using a second seal ring (2) which is conformed in such a way as to be sealedly couplable (for example by interpositioning of a second elastic element) to a portion (20) of the base (T) or the frame (T) when the second part (P2) of the component (P) is mounted and coupled to the base (T) or frame (T).

For example, in the case where the component (P) is constituted by a vibrating cup, as schematically illustrated in figures from 4A to 4D, the first seal ring (1) can be fixed to a portion of the lower edge of the bowl (first part of the component) of the cup which portion surrounds the lower part of the cup (second part of the component) which is destined to be coupled and mounted on a base (T) responsible for driving the cup in vibration.

The second seal ring (2), instead, can be fixed to a portion of the upper edge of the base (T) on which the cup is mounted.

In a case where the method of the invention is to be applied to vibrating cups, already present and used in machines installed in sterile closed environments, the method will comprise adapting the portions of the lower edge of the cup, as well as the portions of the upper edge of the base, so as to receive respectively, in a sealed coupling, the first seal ring and the second seal ring used to clamp the edges of the hole present in the first envelope.

In a case in which, on the other hand, a new machine will have to be designed, to be used in sterile closed environments, then the vibrating cups, and the bases thereof, can be provided for use in this machine already with portions of the relative lower and upper edges with appropriate shapes such as to be able to receive in a sealed coupling the first seal ring and the second seal ring used to clamp the edges of the hole of the first envelope.

FIG. 4A illustrates a vibrating cup (P) which has been wound internally of the first envelope (B1) in such a way that the first part (P1) of the vibrating cup (P) destined for contact with the caps to be used for closing the containers is sealedly closed inside the first envelope (B1), with the second part (P2) of the vibrating cup (P) being positioned at the hole (F) of the first envelope (B1).

The first envelope (B1) is sealedly fixed to a lower portion (10) of the vibrating cup by means of the first seal ring (1) previously applied to the edges of the hole (F).

FIG. 4C is a partial perspective view of FIG. 4A.

FIG. 4B illustrates, in a partial perspective view, the vibrating cup (P) partially wrapped in the first envelope (B1) and completely internal of the second envelope (B2), according to the method of the invention.

FIG. 4D illustrates, in a schematic view in transversal section, the vibrating cup (P), mounted by a coupling of the respective second part (P2) to a base (T) present internally of the closed environment (A) with the first envelope (B1) having been cut, according to the method of the invention.

The second part (P2) of the cup (P) is sealedly coupled to an upper portion (20) of the base (T) or the frame (T) by means of the second seal ring (2) which has been previously applied to the edges of the hole (F) according to the method of the invention.

The same situation is repeated also in a case in which the components to be sterilised are constituted by a hopper (figures from 5A to 5C), or a supply head with dispensing nozzles of liquids or pasty substances (FIGS. 6A-6C).

In these cases too, the method can be applied on hoppers and supply heads already existing in the sector, by adapting the profiles of the respective edges of the parts of the hopper or supply heads destined to be coupled to the profiles of the edges of the heads or frames on which they are to be mounted so as to be able to couplingly receive the first ring and the second seal ring used to clamp the edges of the hole of the first envelope.

Alternatively, during the design step of new machines, the hoppers and supply heads destined to be used in the new machines can also be designed in such a way as already to comprise profiles and edges for couplingly receiving the first ring and the second ring used to clamp the edges of the hole of the first envelope, so as to enable the method of the invention to be carried out.

FIG. 5A illustrates a hopper (P) which is partially wound with a relative first part (P1) internally of the first envelope (B1), and with a relative second part (P2) for coupling with a frame (T) at the hole (F) of the first envelope (B1), and completely closed inside the second envelope (B2), according to the method of the invention.

FIG. 5B illustrates the step of mounting the hopper (P), again wrapped with the respective first part (P1) in the first envelope (B1), on the frame (T), according to the method of the invention, while FIG. 5C illustrates the hopper (P) at the end of the decontamination cycle with the first envelope (B1) having been cut and removed.

FIG. 6A illustrates a supply head with nozzles which is partially wound with a relative first part (P1) internally of the first envelope (B1), and with a relative second part (P2) for coupling with a frame (T) at the position of the hole (F) of the first envelope (B1), and completely closed inside the second envelope (B2), according to the method of the invention.

FIG. 6B illustrates the step of mounting the supply head with nozzles (P), again wrapped with the respective first part (P1) in the first envelope (B1), on the frame (T), according to the method of the invention, while FIG. 6C illustrates the supply head with nozzles (P) at the end of the decontamination cycle, with the first envelope (B1) having been cut and removed.

The method of the invention is however also applicable for the sterilisation of other types of components, as well as those described in the foregoing, used internally of a sterile closed environment of a machine which is to operate in aseptic and sterile conditions so as to carry out the packaging of products, for example pharmaceutical products, internally of relative containers.

In a further possible aspect, on the basis of the type of machine and the modalities used for maintaining the sterile closed environment in class A, the method can comprise carrying out a step of executing (F9) a decontamination cycle internally of the sterile closed environment (FIG. 2H).

For example, before removing the first envelope, the method of the invention can comprise carrying out, internally of the closed environment, a decontamination cycle, for example, though not exclusively, a VHP cycle, in such a way as to decontaminate the outer part of the first closed envelope (B1), as well as the parts surrounding the first envelope at the position of the hole, i.e. at the area in which the second part of the component, which remains exposed with respect to the first envelope, is coupled to the base or frame present internally of the closed environment.

In this way, it is possible to remove all eventual residues of sources of contamination before proceeding with the cutting and removal of the first envelope.

On the basis of a further preferred aspect, in order to carry out the first step of wrapping (F3) the component (P) with the first envelope (B1), the method comprises realising the first envelope (B1) starting from a first film in which a hole (F) is made, the hole being of dimensions suitable for enveloping the second part (P2) of the component (P), or wherein a hole (F) of suitable dimensions for surrounding the second part (P2) of the component is already present.

The first film is applied around the component (P) in such a way that the second part (P2) of the component is positioned at the hole (F), with the film then being sealedly wrapped about the first part (P1) of the component (P) and then closed in such a way as to form the first envelope (B1), with the first part (P1) of the component closed internally thereof and with the second part (P2) of the component (P) remaining exposed and accessible via the hole (F).

In a further aspect, on the other hand, in order to carry out the first step of wrapping (F3) the component (P) with the first envelope (B1), the method can comprise using a first envelope (B1), already formed and having a hole (F), with suitable dimensions for surrounding the second part (P2) of the component, and an open portion for enabling introduction, internally of the first envelope (B1), of the first part (P1) of the component (P), in such a way that the second part (P2) of the component is arranged at the hole (F), and then closing the open portion of the first envelope (B1) to enclose internally thereof and insulate the first part (P1) of the component (P) from the outside environment.

In a possible further aspect, for the execution of the step of demounting (F1) of the component (P) from the base (T) or the frame (T) of the machine (M), the method can comprise, once the component (P) has been demounted from the base (T) or from the frame of the machine (M), inserting the component (P) in a protective casing (H) (see the illustration on the left of FIG. 2B) for containing internally thereof any dangerous substances remaining in contact with the component (P), and removing the protective casing (H) (see the upper illustration in FIG. 2C) before inserting the component (P) in the washing machine.

According to the methods used for carrying out the sterilisation step, the method preferably comprises using, or realising, the first envelope (B1) and the second envelope (B2) in a multilayer material, or composed by materials having properties of permeability to the passage of fluid used for sterilisation.

In a further advantageous and preferred aspect, the method can comprise carrying out the step of cutting and removal (F10) of the first envelope (B1) from the first part (P1) of the component (P) automatically by using cutting and removing means (30) which are mounted at the base (T) or frame (T) of the machine (M) internally of the closed environment (A), and which are activatable to remove portions of the first envelope (B1) once the second part (P2) of the component (P) has been mounted and coupled to the base (T) or frame (T).

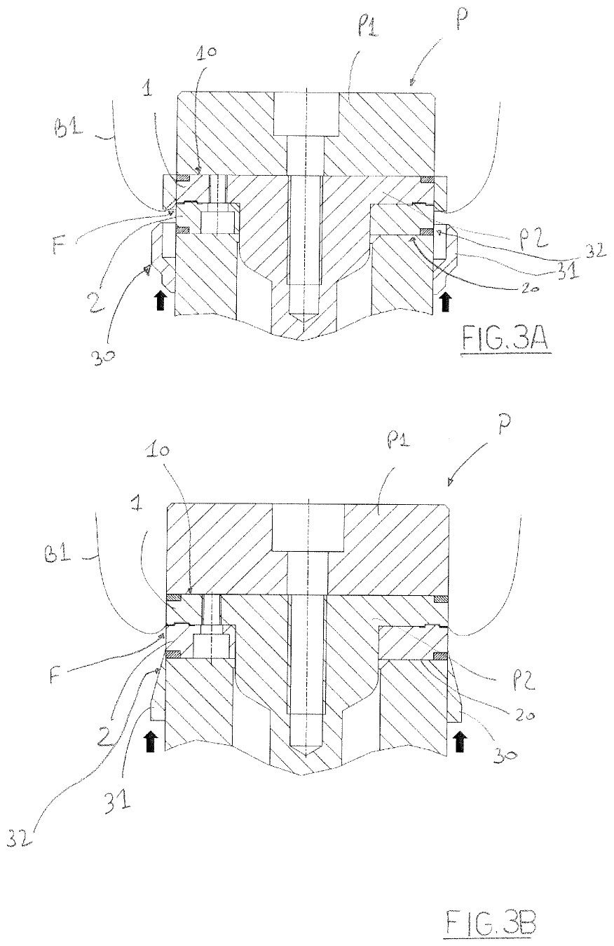

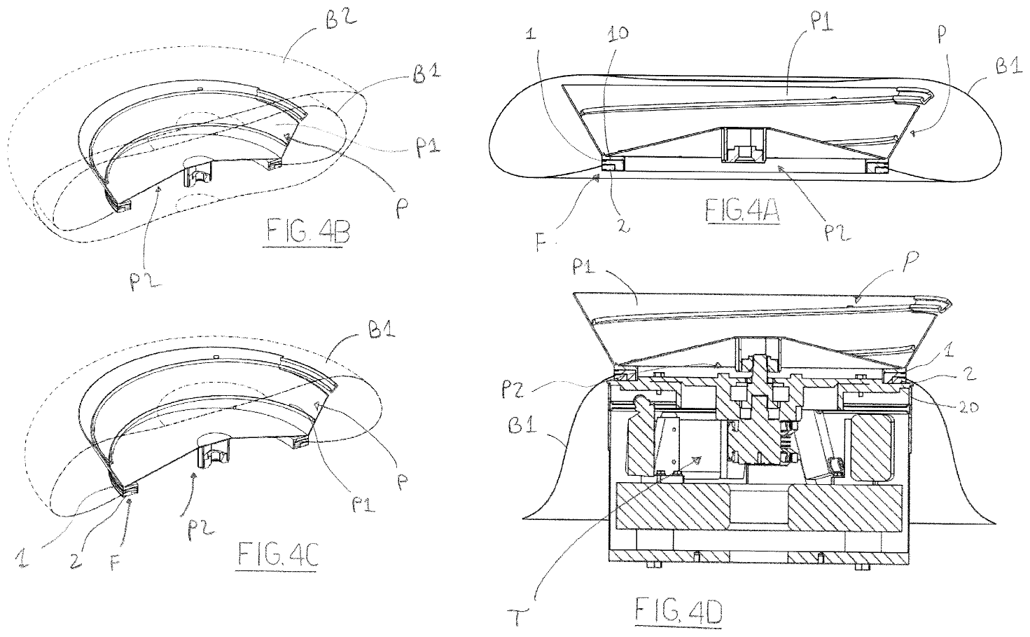

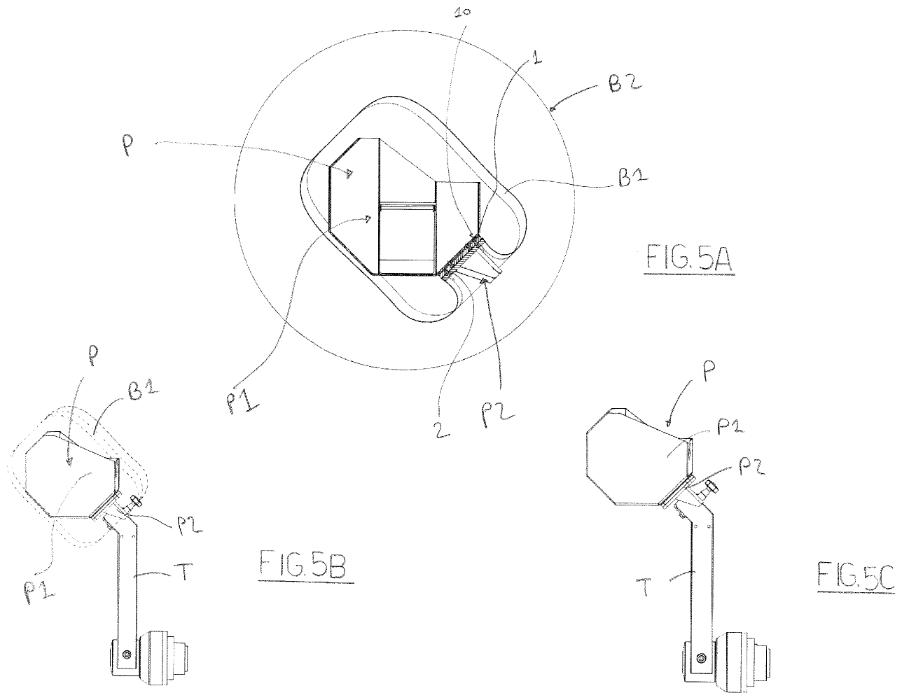

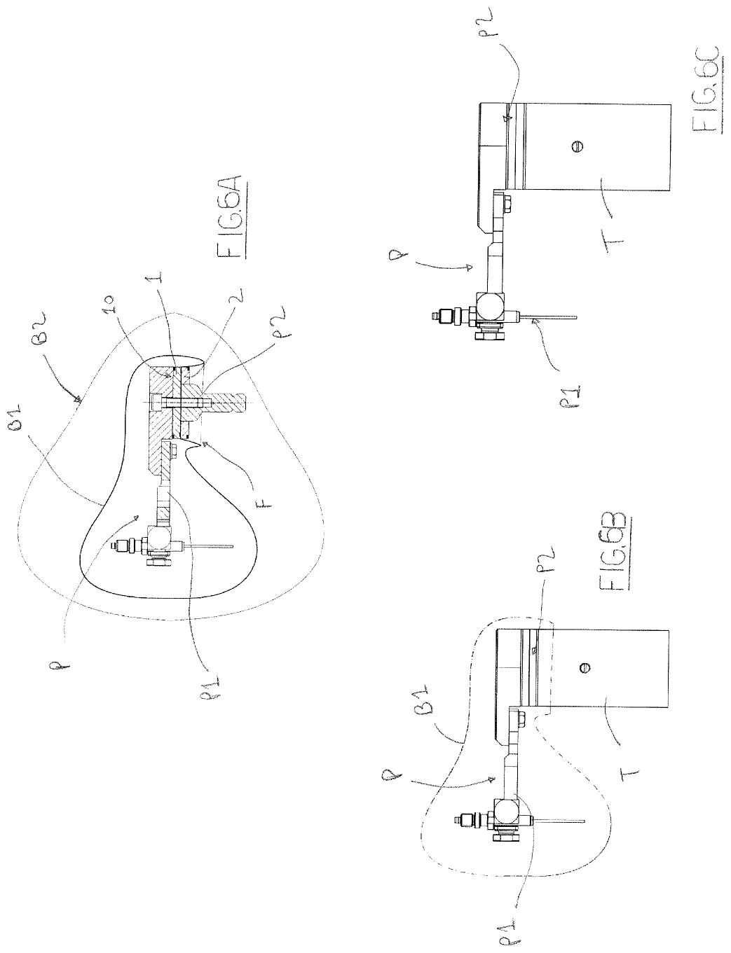

For example, the method can comprise using cutting and removing means (30) comprising at least a sliding element (31), which is movably mounted on the base (T) or frame (T), and has a cutting profile (32) (see for example two possible modalities of realisation of the sliding element illustrated in FIG. 3A, in which the cutting profile is constituted by a hollow element which couples with an abutment for clamping portions of the envelope and cutting them, or in FIG. 3B in which the cutting profile is constituted by a blade).

Once the second part (P2) of the component (P) has been mounted and coupled to the base (T) or frame (T), the sliding element (31) is drivable in translation with respect to the base (T) or frame (T) in such a way that the cutting profile (32) abuts and cuts portions of the first envelope (B1) in proximity of the edges of the hole (F).

Therefore, in conclusion, the method of the invention enables carrying out the operations of sterilisation of the components destined for direct contact with the products to be processed in sterile closed environments and remounting the components internally of the sterile closed environments, while safeguarding and protecting from any contamination the parts of the components destined for contact with the products, as they are sealedly closed internally of the first envelope up to when the component has been mounted on the relative base or frame present in the closed environment.

Only at this point is the first envelope removed, freeing the first part of the component contained therein which will be entirely sterile and ready to be used for a new productive cycle internally of the sterile closed environment.

1. A method for sterilising a component of a machine operating in a sterile closed environment and used for contact with products which are to be processed internally of the sterile closed environment,
wherein the component that is to be sterilised comprises at least a first part, destined for direct contact with the products to be processed internally of the sterile closed environment, and at least a second part, conformed for coupling and mounting on a base or on a frame of the machine present internally of the sterile closed environment and comprising driving means for driving and operating the component,
wherein the method comprises:
a step of demounting the component from the base or from the frame of the machine present internally of the closed environment;
a first step of transferring the component to outside the sterile closed environment and inserting the component internally of a washing machine in order to carry out a washing cycle of the component;
wherein once the washing cycle has finished, the method further comprises:
a first step of wrapping of the component with a first envelope comprising completely wrapping the first part of the component destined for direct contact with the products leaving the second part of the component, destined for coupling and mounting on the base or the frame present internally of the sterile closed environment, exposed and accessible via a hole present in the first envelope, wherein the first step of wrapping comprises coupling edges of the hole sealingly to a portion of the component between the first part and the second part of the component to isolate the first part of the component from an outside environment;
a second step of wrapping comprising completely wrapping the first envelope, and the second part of the component which remains exposed from the first envelope, with a second envelope in such a way that the first envelope, and the second part of the component which is exposed and accessible from the first envelope via the hole, are completely closed internally of the second envelope, with the second envelope being closed;
a second step of transferring of the component, partially wrapped in the first envelope and completely contained in the second closed envelope, internally of a sterilisation machine in order to carry out a sterilisation cycle by means of a sterilisation flow, wherein the first envelope and the second envelope are made of a material capable of allowing passage of the sterilisation flow in such a way that the sterilisation flow can sterilise both the second part of the component, exposed and accessible via the first envelope via the hole, and arranged internally of the second envelope, and also the first part of the component closed internally of the first envelope;
wherein, once the sterilisation cycle has finished, the method further comprises:
a third step of transferring the component with the first part wrapped in the first envelope and completely closed in the second envelope in a position facing the sterile closed environment;
a step of cutting and removal of the second envelope;
a fourth step of transferring the component, with the first envelope still being sealedly wrapped about the first part of the component and with the second part of the component being exposed and accessible via the hole of the first envelope, internally of the sterile closed environment, and mounting the second part of the component on the base or the frame present internally of the sterile closed environment, maintaining the first part of the component sealedly closed in the first envelope;
a step of cutting and removal of the first envelope from the first part of the component.

2. The method as claimed in claim 1, comprising using a first seal ring and a second seal ring and applying the first seal ring and the second seal ring on opposite sides of the hole to clamp there-between portions of the first envelope which surround the hole, wherein the first seal ring is conformed in such a way as to be sealedly coupled to a portion of the component between the first part of the component and the second part of the component, and wherein the second seal ring is conformed in such a way as to be sealedly coupled to a portion of the base or the frame when the second part of the component is mounted and coupled to the base or the frame.

3. The method as claimed in claim 1, wherein the first step of wrapping the component with the first envelope comprises realising the first envelope starting from a first film in which a hole is made, the hole being of dimensions suitable for enveloping the second part of the component, or wherein a hole of suitable dimensions for surrounding the second part of the component is already present, with the first film being applied around the component in such a way that the second part of the component is positioned at the hole, with the film then being folded about the first part of the component and then closed in such a way as to form the first envelope, with the first part of the component closed internally thereof and with the second part of the component remaining exposed and accessible via the hole.

4. The method as claimed in claim 1, wherein the first step of wrapping the component with the first envelope comprises using a first envelope already formed and having a hole, having suitable dimensions for surrounding the second part of the component, and an open portion for enabling introduction internally of the first envelope of the first part of the component in such a way that the second part of the component is arranged at the hole, and then closing the open portion of the first envelope to close internally thereof, and insulate, from outside, the first part of the component.

5. The method as claimed in claim 1, wherein the step of cutting and removal of the first envelope from the first part of the component takes place automatically by using cutting and removing means which are mounted at the base or the frame of the machine internally of the sterile closed environment and are activatable to remove portions of the first envelope once the second part of the component has been mounted and coupled to the base or the frame.

6. The method as claimed in claim 5, wherein the step of cutting and removal of the first envelope takes place using cutting and removing means comprising at least a sliding element, movably mounted on the base or the frame, and having a cutting profile, wherein the sliding element, once the second part of the component has been mounted and coupled to the base or the frame, is drivable in alternating translation with respect to the base or the frame in such a way that the cutting profile abuts and cuts portions of the first envelope in proximity of the edges of the hole.

7. The method as claimed in claim 1, comprising carrying out a step of executing a decontamination cycle internally of the sterile closed environment before carrying out the step of cutting and removal of the first envelope from the first part of the component.
